(19) United States
(12) Patent Application Publication
(10) Pub. No.: US 2011/0178470 A1
(43) Pub. Date: Jul. 21, 2011

(54) PHARMACEUTICAL COMPOSITIONS COMPRISING BORONIC ACID COMPOUNDS

(75) Inventors: Chandrasekhar Kocherlakota, Secunderabad (IN); Lavanya Nallamothu, Hyderabad (IN); Krishnam Raju Kovoru, East Godavari District (IN); Nagaraju Banda, Hyderabad (IN); Mittapally Sridhar, Karimnagar (IN); Keni Devendra Chandrkanth, Mumbai (IN); Soogareddy Channareddy Shantreddy, Hyderabad (IN); Wagh Sanjay Chhagan, Hyderabad (IN); Pillai Raviraj Sukumar, Hyderabad (IN); Bandari Sreedhar, Karimnager (IN); Hinge Kranthikumar, Warangal (IN)

(73) Assignees: DR. REDDY'S LABORATORIES LTD., Hyderabad (IN); DR. REDDY'S LABORATORIES, INC., Bridgewater, NJ (US)

(21) Appl. No.: 13/076,839

(22) Filed: Mar. 31, 2011

Related U.S. Application Data
(63) Continuation of application No. PCT/US2009/058929, filed on Sep. 30, 2009.
(60) Provisional application No. 61/141,287, filed on Dec. 30, 2008.
(60) Provisional application No. 61/148,555, filed on Jan. 30, 2009.
(60) Provisional application No. 61/185,263, filed on Jun. 9, 2009.

(30) Foreign Application Priority Data
Oct. 1, 2008 (IN) 2415/CHE/2008
Dec. 16, 2008 (IN) 3160/CHE/2008
Feb. 18, 2009 (IN) 363/CHE/2009

(51) Int. Cl. A61K 31/69; A61P 35/00; A61M 5/178; A61J 1/05

(52) U.S. Cl. 604/187; 514/64; 604/403

(57) ABSTRACT

Pharmaceutical compositions comprising bortezomib for oral or parenteral administration. Specific aspects relate to stable, sugar free pharmaceutical compositions of bortezomib, including its pharmaceutically acceptable salts or solvates, in the form of ready-to-use solutions, lyophilized forms, or physical admixtures, and the preparation thereof. Other aspects include processes for preparing compositions and methods of using compositions for treating various types of cancers in mammals.

### INTRODUCTION

Aspects of the present invention relate to pharmaceutical compositions comprising boronic acid compounds or modified boronic acid compounds. Further aspects of the present invention relate to pharmaceutical compositions for oral or parenteral administration comprising bortezomib, including its pharmaceutically acceptable salts or solvates. Also included are processes for preparing such compositions and methods of using such compositions for treating various types of cancers in mammals.

A boronic acid is an alkyl or aryl substituted boric acid containing a carbon-to-boron chemical bond, belonging to the larger class of organo-boranes. Boronic acids act as Lewis acids. They have the unique feature of being capable of forming reversible covalent complexes with sugars, amino acids, hydroxamic acids, etc. (molecules with vicinal, (1,2-) or occasionally (1,3-) substituted Lewis base donors (alcohol, amine, carboxylate). The pKₐ of a boronic acid is about 9, but upon complexion in aqueous solutions they form tetrahedral boronate complexes with pKₐ about 7.

Boronic acid and ester compounds display a variety of pharmaceutically useful biological activities. Shenvi et al., U.S. Pat. No. 4,499,082 (1985), discloses that peptide boronic acids are inhibitors of certain proteolytic enzymes. Kettner and Shenvi, U.S. Pat. No. 5,187,157 (1993), U.S. Pat. No. 5,242,904 (1993), and U.S. Pat. No. 5,250,720 (1993), describe a class of peptide boronic acids that inhibit trypsin-like proteases. Kleeman et al., U.S. Pat. No. 5,169,841 (1992), discloses N-terminally modified peptide boronic acids that inhibit the action of renin. Kinder et al., U.S. Pat. No. 5,106,948 (1992), discloses that certain tripeptide boronic acid compounds inhibit the growth of cancer cells.

Unfortunately, alkylboronic acids are relatively difficult to obtain in analytically pure form. H. R. Snyder et al., “Aryl Boronic Acids. II. Aryl Boronic Anhydrides and their Amine Complexes,” Journal of the American Chemical Society, Vol. 80, 3611-3615 (1958), teaches that alkylboronic acid compounds readily form boroxines (anhydrides) under dehydrating conditions. Also, alkylboronic acids and their boroxines are often air-sensitive. S. Korcek et al., “Absolute Rate Constants for the Autoxidation of Organometallic Compounds. Part II. Benzylboranes and 1-Phenylethylboranes,” Journal of the Chemical Society, Perkin Transactions 2, pp. 242-248 (1972), teaches that butylboronic acid is readily oxidized by air to generate 1-butanol and boric acid. These difficulties limit the pharmaceutical utility of boronic acid compounds, complicating the characterization of pharmaceutical agents comprising boronic acid compounds and limiting their shelf life.

There is a need to prepare improved and stable formulations of boronic acid compounds. Ideally, such formulations would be conveniently prepared, would exhibit enhanced stability and longer shelf life as compared to the other boronic acid compound, and would readily liberate the bioactive boronic acid compound when administered to a subject in need of boronic acid therapy.

Bortezomib is a modified di-peptidyl boronic acid. It is the first therapeutic proteasome inhibitor to be tested in humans. The product is provided commercially as a mannitol boronic ester which, in reconstituted form, consists of the mannitol ester in equilibrium with its hydrolysis product, the monomeric boronic acid. The drug substance exists in its cyclic anhydride form as a trimeric boroxine.

The chemical name for bortezomib, the monomeric boronic acid, is [(1R)-3-methyl-1-[[(2S)-1-oxo-3-phenyl-2-[(pyrazinylcarbonyl)amino]propyl]amino]butyl]boronic acid. The solubility of bortezomib, as the monomeric boronic acid, in water is 3.3 to 3.8 mg/mL over a pH range of 2 to 6.5. Bortezomib has the following chemical structure.

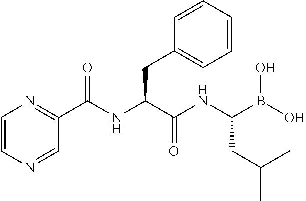

Bortezomib is a reversible inhibitor of the chymotrypsin-like activity of the 26S proteasome in mammalian cells. The 26S proteasome is a large protein complex that degrades ubiquitinated proteins. The ubiquitin-proteasome pathway plays an essential role in regulating the intracellular concentration of specific proteins, thereby maintaining homeostasis within cells. Inhibition of the 26S proteasome prevents this targeted proteolysis, which can affect multiple signaling cascades within the cell. This disruption of normal homeostatic mechanisms can lead to cell death.

Commercially, bortezomib is available in a product sold as VELCADE® sterile lyophilized powder for intravenous infusion and available in single-dose vials. Each single dose vial contains 3.5 mg of bortezomib as a sterile lyophilized powder. The inactive ingredient is 35 mg mannitol, USP, per vial.

U.S. Pat. Nos. 6,699,835, 6,713,446, 6,958,319, and 7,109,323 describe stable pharmaceutical compositions of boronic acid compounds which are prepared by lyophilizing an aqueous mixture comprising a boronic acid compound and a compound having at least two hydroxyl groups which produces a stable composition that readily releases the boronic acid compound upon dissolution in aqueous media.

International Application Publication No. WO 2006/063154 describes pharmaceutical compositions comprising a practically insoluble proteasome inhibitor and a cyclodextrin. The invention uses cyclodextrins to increase the solubility of the practically insoluble proteasome inhibitors. But the invention is restricted to proteasome inhibitors such as peptide epoxy ketones which are more highly proteasome-specific inhibitors that could have fewer toxic side effects when compared to other proteasome inhibitors such as bortezomib. More specifically the application excludes bortezomib and restricts the invention to peptide epoxy ketones.

However, formulating bortezomib has not proven to be an easy task, typically due to stability and solubility issues. There remains a need for preparing bortezomib formulations with improved stability and increased solubility.

There also exists an immediate need for making stable bortezomib compositions for parenteral and oral administration, in addition to the existing lyophilized formulations of bortezomib, as lyophilization process involves high capital costs of equipment, high energy costs and long processing times (typically a 24-hour drying cycle).

### SUMMARY

Aspects of the present invention relate to pharmaceutical compositions comprising bortezomib for oral or parenteral administration. In embodiments, the invention relates to stable sugar free pharmaceutical compositions of bortezomib, including its pharmaceutically acceptable salts or solvates, in the form of ready-to-use solutions or lyophilized forms or physical admixtures and preparations thereof. Other aspects include processes for preparing such compositions and methods of using such compositions for treating various types of cancers in mammals.

An aspect of the present invention provides sugar free physical admixtures, lyophilized preparations, or ready-to-use solutions, comprising bortezomib and optionally a stabilizing agent, which produce stable compositions.

Another aspect of the invention provides physical admixtures, lyophilized preparations, or ready-to-use solutions comprising bortezomib and sodium chloride, a vitamin, a carboxylic acid, or an amino acid; and optionally a stabilizing agent, which produces stable compositions that readily release the boronic acid compound upon dissolution in aqueous media.

An aspect of the invention provides methods of producing stable pharmaceutical products comprising lyophilized bortezomib, which methods comprise preparing a composition comprising bortezomib and a solvent, which solvent comprises at least one alcohol.

An aspect of the present invention provides bortezomib preparations comprising bortezomib and optionally t-butyl alcohol, which are lyophilized to produce stable bortezomib lyophilized preparations.

An aspect of the present invention provides lyophilized bortezomib preparations which are stable in closed containers for at least one week at 60° C.

Another aspect of the invention provides pharmaceutical compositions comprising bortezomib and a solubilizer or a cyclodextrin, for oral administration.

An aspect of the invention provides physical admixtures, lyophilized preparations, and ready-to-use solutions comprising bortezomib and a cyclodextrin or a solubilizer for parenteral administration.

### BRIEF DESCRIPTION OF THE DRAWING

FIG. 1 is a flow diagram that schematically describes a process for obtaining lyophilized preparations comprising bortezomib.

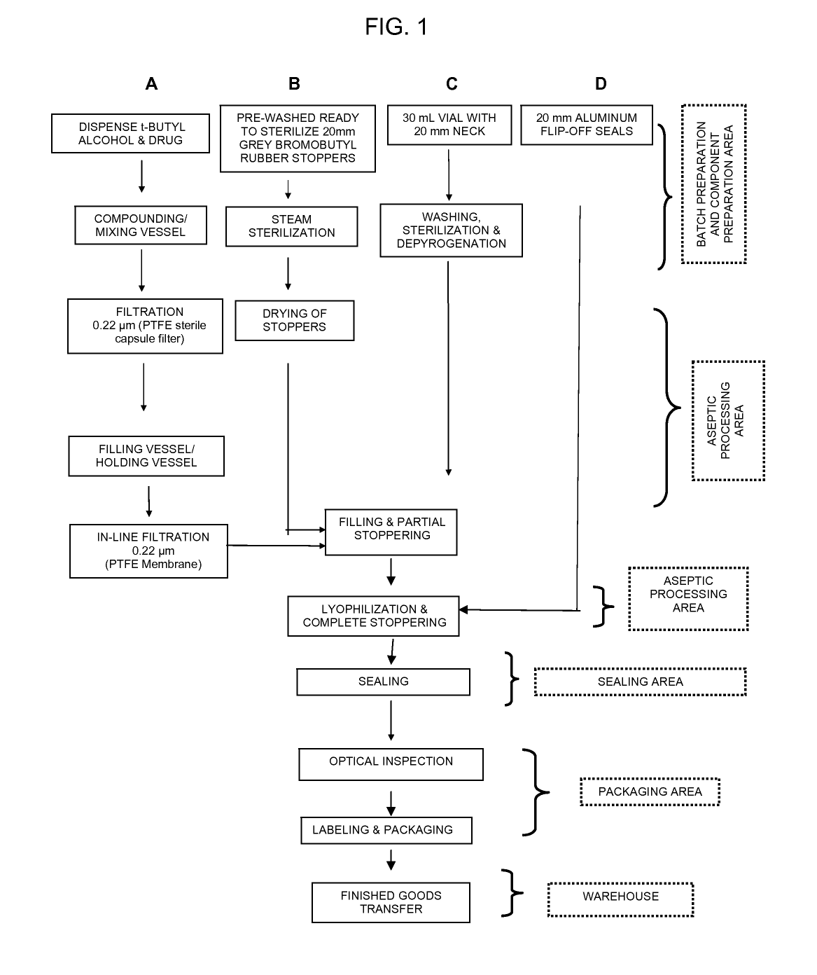

### DETAILED DESCRIPTION

Aspects of the present invention relate to pharmaceutical compositions comprising bortezomib for oral or parenteral administration. In specific aspects, the invention relates to stable sugar free pharmaceutical compositions comprising bortezomib, including its pharmaceutically acceptable salts or solvates, in the form of ready-to-use solutions, lyophilized forms, or physical admixtures, and preparations thereof. Other aspects include processes for preparing such compositions and methods of using such compositions for treating various types of cancers in mammals.

The term “pharmaceutically acceptable” refers to substances that do not have inherent pharmacological activity and are used as inactive ingredients. Such substances are generally safe, non-toxic and neither biologically nor otherwise undesirable, and include those acceptable for veterinary use as well as human pharmaceutical use.

As used herein, the terms “composition” and “formulation” refer to preparations comprising a boronic acid compound in a form suitable for administration to a human or other mammal.

The term “sugar free” refers to compositions that are substantially free of sugars during preparation, after preparation, or anytime during the manufacturing process of a composition. The term “sugar” refers to any carbohydrate material specifically relating to a disaccharide, monosaccharide, or sugar alcohol, non limiting examples including dextrose, mannose, lactose, mannitol, sorbitol, sucrose, and artificial sugars like natural or synthetic sweeteners including sorbitol, saccharose, saccharine, aspartame, acelsulphame K, cyclamate, and the like.

The term “diol free” refers to compositions that are substantially free of diol compounds during preparation, after preparation, or at any time during the manufacturing process of a composition. The term “diol” refers to any diol, such as pinanediol, pinacol, perfluoropinacol, ethylene glycol, diethylene glycol, catechol, 1,2-cyclohexanediol, 1,3-propanediol, 2,3-butanediol, 1,2-butanediol, 1,4-butanediol, and glycerol.

The term “stable” refers to bortezomib preparations having sufficient stability to allow storage at a commercially relevant temperature, such as between about 0° C. and about 60° C., for a commercially relevant period of time, such as at least one week, one month, three months, six months, one year, or two years.

The term “admixture” refers to any mixture of solid material obtained by physical mixing, which may or may not employ a mechanical device.

The term “alcohol” refers to an organic compound having a free ‘OH’ hydroxy group, and is used for dissolving active agent and as a solvent for lyophilization.

The term “solvent” refers to an ingredient used for dissolving an ingredient.

The term “boronic acid” is intended to encompass free boronic acid compounds, oligomeric anhydrides, dimers, trimers, and tetramers, ester derivatives with amino acids, peptides, and mixtures thereof in general, as well as isosteric variations thereof and their compositions.

“Solubilizer” refers to any substance which enhances the aqueous solubility of a drug. Solubilizers can be surface active agents (also known as “surfactants”), co-solvents, and complexing agents.

The term “amino acid” as used in some embodiments includes, without limitation thereto, α-amino acids such as lysine, arginine, glutamine, asparagine, threonine, serine, and the like.

The term “vitamin” as used in some embodiments includes, without limitation thereto, thiamine, folic acid, nicotinic acid, nicotinamide, and the like.

The term “carboxylic acid” as used in some embodiments includes, without limitation thereto, citric acid, malic acid, succinic acid, and the like.

The term “stabilizing agent” identifies an agent which improves the composition stability for a reasonable period of time, such as those mentioned above, at certain temperatures. A stabilizing agent may or may not be included in the compositions. Stabilizing agents in the compositions include, but are not limited to, ethylenetetraamineacetic acid, ethylenediaminetetraacetic acid (EDTA), and salts thereof.

The compositions of the present invention can be administered in any form. Examples include, but are not limited to, creams, gels, solutions, suspensions, liposomes, particles, or other means known to one of skill in the art of formulation and delivery of therapeutic and cosmetic compounds. Some examples of appropriate formulations for subcutaneous administration include but are not limited to implants, depots, capsules, and osmotic pumps. Some examples of appropriate formulations for vaginal administration include but are not limited to creams and rings. Some examples of appropriate formulations for transdermal injectable formulations are typically formulated as aqueous solutions in which water is the primary excipient. Injectable formulations can be prepared in conventional forms, either as liquid solutions or suspensions, solid forms suitable for solubilization or suspending in liquid prior to injection, or as emulsions. Sterile injectable formulations can be prepared according to techniques known in the art using suitable carriers, dispersing or wetting agents, and/or suspending agents. The injectable formulations may be sterile injectable solutions or suspensions in a nontoxic, parenterally acceptable diluent or solvent. Among the acceptable vehicles and solvents that may be employed are water, Ringer's solution, and isotonic sodium chloride solution. In addition, fixed oils, fatty esters or polyols are conventionally employed as solvents or suspending media.

The injectable pharmaceutical formulations may optionally include one or more pharmaceutically acceptable excipients. The pharmaceutically acceptable excipients may include any one or more of: one or more antibacterial preservatives, including one or more of phenylmercuric nitrate, thiomersal, benzalkonium chloride, benzethonium chloride, phenol, cresol and chlorobutanol; antioxidants including one or more of ascorbic acid, sodium sulfite, sodium bisulfite and sodium metabisulfite; buffers including one or more of acetate, citrate, tartarate, phosphate, benzoate and bicarbonate buffers; and tonicity contributors including one or more of sodium chloride, potassium chloride, and alkaline substances including one or more of sodium hydroxide, potassium hydroxide, sodium carbonate and meglumine and salts such as sodium chloride.

The amount of bortezomib that can be solubilized is dependent on several parameters. One such parameter is pH. Higher pH results in poorer solubility of a basic compound, and lower pH would be expected to decrease solubility of an acidic compound, as is known in the art. However, a pH should be selected to provide suitable stability of the proteasome inhibitor. For formulations to be administered to a mammal, the pH is frequently from about 2.5 to about 9.

A primary source of pH control can be a buffer. Typically, a buffer is present as an acid or a base and its conjugate base or acid, respectively. In one embodiment, the concentration of buffering salt in a solution is about 1-100 mM, or about 5-50 mM, or about 10 mM. In solid formulations, the amount of buffer is selected to produce this concentration after reconstitution/dilution. The concentration of buffer and the pH of the solution are advantageously chosen to give an optimal balance of solubility and stability. Examples of suitable buffers include mixtures of weak acids and alkali metal salts (e.g., sodium, potassium) of the weak acids, such as sodium tartrate and sodium citrate.

Solubilizers can provide pharmaceutical compositions comprising bortezomib that show a nearly constant rate of drug absorption and concurrently maintain a high extent of bioavailability. This objective is achieved by using a solubilizer which is mixed intimately with the drug. The active compound is dissolved or dispersed in the solubilizer. The mixture of pharmaceutically active compound and solubilizer can be diluted with water or intestinal fluids without significant precipitation of the dissolved drug. In a solution, the drug can be included in a micelle structure formed by the solubilizer.

A variety of suitable solubilizers may be used, as long as the aqueous solubility of the drug is increased. Examples of solubilizers are polyoxyethylene-polyoxypropylene (POE-POP) block copolymers, fatty alcohols and fatty alcohol derivatives, and acids, particularly fatty acids and fatty acid derivatives and tocol derivatives. Useful fatty acids and alcohols include the C₆-C₂₂ fatty acids and C₈-C₂₂ alcohols, capric acid, caprylic acid, lauric acid, myristic acid, stearic acid, oleic acid, linoleic acid, linolenic acid, arachidonic acid, behenic acid, and their corresponding pharmaceutically acceptable salts. Examples of fatty acid and fatty alcohol derivatives include sodium dioctyl sulfosuccinate, sodium lauryl sulfate, amide esters (e.g., lauric acid diethanolamide, sodium lauryl sarcosinate, lauroyl carnitine, palmitoyl carnitine, and myristoyl carnitine), esters with hydroxy-acids (e.g., sodium stearoyl lactylate); sugar esters, e.g., lauryl lactate, glucose monocaprylate, diglucose monocaprylate, sucrose laurate, sorbitan monolaurate (Arlacel® 20), sorbitan monopalmitate (Span® 40), sorbitan monooleate (Span 80), lower alcohol fatty acid esters e.g., ethyl oleate (Crodamol® EO), isopropyl myristate (Crodamol IPM) and isopropyl palmitate (Crodamol IPP), esters with propylene glycol [e.g., propylene glycol monolaurate (Lauroglycol™ FCC), propylene glycol ricinoleate (Propymuls®), propylene glycol monooleate (Myverol® P-06), propylene glycol monocaprylate (Capryol® 90), propylene glycol dicaprylate/dicaprate (Captex® 200) and propylene glycol dioctanoate (Captex 800), esters with glycerol e.g., glyceryl monooleate, glyceryl ricinoleate, glyceryl laurate, glyceryl dilaurate (Capmul® GDL), glyceryl dioleate (Capmul GDO), glycerol monolinoleate (Maisine®), glyceryl mono/dioleate (Capmul GMO-K), glyceryl caprylate/caprate (Capmul MCM), caprylic acid mono/diglycerides (Imwitor® 988), mono- and di-acetylated monoglycerides (Myvacet® 9-45), triglycerides, e.g., corn oil, almond oil, soybean oil, coconut oil, castor oil, hydrogenated castor oil, hydrogenated coconut oil, Pureco 100, Hydrokote AP5, Captex 300, 350, Miglyol® 812, Miglyol 818 and Gelucire® 33/01), mixtures of propylene glycol esters and glycerol esters e.g., mixtures of oleic acid esters of propylene glycol and glycerol (Arlacel 186), and polyglycerized fatty acids such as polyglyceryl oleate (Plurol® Oleique), polyglyceryl-2 dioleate (Nikko DGDO), polyglyceryl-10 trioleate, polyglyceryl-10 laurate (Nikkol Decaglyn 1-L), polyglyceryl-10 oleate (Nildcol Decaglyn 1-0), and polyglyceryl-10 mono dioleate (Caprol® PEG 860).

Other useful fatty acid derivatives include polyethoxylated fatty acids, (e.g., PEG-8 laurate, PEG-8 oleate, PEG-8 stearate, PEG-9 oleate, PEG-1O laurate, PEG-1O oleate, PEG-12 laurate, PEG-12 oleate, PEG-15 oleate, PEG-20 laurate and PEG-20 oleate), PEG-fatty acid diesters (e.g., PEG-20 dilaurate, PEG-20 dioleate, PEG-20 distearate, PEG-32 dilaurate and PEG-32 dioleate), PEG-fatty acid mono- and di-ester mixtures, polyethylene glycol glycerol fatty acid esters (e.g., PEGylated glycerol 12 acyloxy-stearate, PEG-20 glyceryl laurate, PEG-30 glyceryl laurate, PEG-40 glyceryl laurate, PEG-20 glyceryl oleate and PEG-30 glyceryl oleate) and alcohol-oil transesterification products [e.g., polyoxyl 40 castor oil (Cremophor® RH40), polyoxyl 35 castor oil (Cremophor EL or Incrocas 35), PEG-25 trioleate (TAGAT® TO), PEG-60 corn glycerides (Crovol M70), PEG-60 almond oil (Crovol A70), PEG 40 palm kernel oil (Crovol PK70), PEG-50 castor oil (Emalex C-50), PEG-50 hydrogenated castor oil (Emalex HC-50), PEG-60 hydrogenated castor oil (Cremophor RH60), PEG-8 caprylic/capric glycerides (Labrasol®), lauroyl macrogol 32 glycerides (Gelucire© 44/14), linoleoyl macrogol glycerides (Labrafil®), stearoyl macrogol-32 glycerides (Gelucire 50/13), and PEG-6 caprylic/capric glycerides (Softigen® 767).

Cyclodextrins (CDs) are cyclic oligomers of glucose, which typically contain 6, 7, or 8 glucose monomers joined by α-1, 4 linkages. These oligomers are commonly called α-CD, β-CD, and γ-CD, respectively. Higher oligomers containing up to 12 glucose monomers are also known. Topologically, CDs can be represented as a toroid in which the primary hydroxyls are located on the smaller circumference, and the secondary hydroxyls are located on the larger circumference. Because of this arrangement, the interior of the torus is hydrophobic while the exterior is sufficiently hydrophilic to allow the CD to be dissolved in water. This difference between the interior and exterior faces allows the CD to act as a host molecule and to form inclusion complexes with guest molecules, provided that the guest molecule is of the proper size to fit in the cavity. The CD inclusion complex can then be dissolved in water thereby providing for the introduction of guest molecule that has little or no aqueous solubility into an aqueous environment. Reviews of CD complexes include K. A. Connors, “The Stability of Cyclodextrin Complexes in Solution,” Chemical Reviews, Vol. 97 (5), pages 1325-1357 (1997), and J. Szejtli, “Selectivity/Structure Correlation in Cyclodextrin Chemistry,” Supramolecular Chemistry, Vol. 6 (1 and 2), pages 217-223 (1995).

Unmodified cyclodextrins, especially β-cyclodextrin, have limited aqueous solubility, have relative large molecular weights, and tend to crystallize from solution. The combination of these issues means that their ability to solubilize and stabilize guest molecules in an aqueous environment is limited. Additionally, unmodified cyclodextrins, e.g. β-cyclodextrin, have been shown to cause renal and liver damage after parenteral administration. These issues have led to exploration of the use of chemically modified or derivatized cyclodextrins that avoid some of these problems. Two examples of derivatized cyclodextrins are hydroxybutenyl cyclodextrins (HBenCD), which are disclosed in U.S. Pat. No. 6,479,467 (2002) and in C. M. Buchanan et al., “Synthesis and Characterization of Water-Soluble Hydroxybutenyl Cyclomaltooligosaccharides (Cyclodextrins),” Carbohydrate Research, Vol. 337 (6), pages 493-507 (2002), and sulfonated hydroxybutenyl cyclodextrins (SulfoHBenCD), which are disclosed in U.S. Pat. No. 6,610,671.

Cyclodextrins that are useful in the present invention include alpha-, beta- and gamma-cyclodextrins. In embodiments, the cyclodextrin is either a substituted or non-substituted 3-cyclodextrin. Examples of substituted 3-cyclodextrins include those substituted with one or more hydrophilic groups, such as monosaccharide (e.g., glucosyl, maltosyl), carboxyalkyl (e.g., carboxylmethyl, carboxyethyl), hydroxyalkyl-substituted (e.g., hydroxyethyl, 2-hydroxypropyl) and sulfoalkylether-substituted beta-cyclodextrin. Particularly suitable beta-cyclodextrins include hydroxypropyl beta-cyclodextrin (HPBCD) and sulfobutylether beta-cyclodextrin (SBECD). However, it is understood that typically any substitution to the cyclodextrin, including substitution by hydrophobic groups such as alkyl groups, will improve its aqueous solubility by disrupting the hydrogen-bonding network within the crystal lattice of the solid cyclodextrin, thereby lowering the lattice energy of the solid. The degree of substitution is not believed to be critical; however, the degree of substitution is advantageously at least about 1%, and typically about 2% to 10%, such as about 3% to 6%.

The CD derivatives serve to solubilize and stabilize the pharmaceutically active compound when the composition is added to an aqueous environment as well as provide for enhanced and/or sustained release and to increase bioavailability in the appropriate physiological environment.

In embodiments of the invention, bortezomib and cyclodextrin are present in mixtures in molar ratios ranging from about 0.5:1 to about 100:1, or from about 5:1 to about 100:1.

In embodiments of the invention, bortezomib and a solubilizer or combination of solubilizers are present in the mixture in molar ratios ranging from about 0.5:1 to about 100:1, or from about 5:1 to about 100:1.

In one embodiment, the invention provides physical admixtures, ready-to-use solutions, or lyophilized preparations comprising bortezomib or a pharmaceutically acceptable salt or solvate thereof; and a pharmaceutically acceptable carrier, wherein the pharmaceutically acceptable carrier comprises a cyclodextrin or a solubilizer.

In embodiments, the invention provides pharmaceutical compositions for oral administration, comprising bortezomib or a pharmaceutically acceptable salt or solvate thereof; and a pharmaceutically acceptable carrier, wherein the pharmaceutically acceptable carrier comprises a cyclodextrin or a solubilizer.

In embodiments of the invention, bortezomib and an amino acid, vitamin, carboxylic acid, sodium chloride, or stabilizing agent are present in the mixture in molar ratios ranging from about 0.5:1 to about 100:1, or from about 5:1 to about 100:1. In some aspects of the invention, bortezomib and amino acid, vitamin, carboxylic acid, sodium chloride, or stabilizing agent are present in molar ratios ranging from about 10:1 to about 100:1.

In certain embodiments, the invention provides sugar free or diol free compositions comprising bortezomib or a pharmaceutically acceptable salt or solvate thereof; and a pharmaceutically acceptable carrier, wherein the pharmaceutically acceptable carrier comprises at least one of an amino acid, vitamin, carboxylic acid, and sodium chloride, and may, or may not, comprise a stabilizing agent.

In the above embodiment, the composition may be in the form of a physical admixture, a lyophilized preparation, or a ready-to-use solution.

In certain embodiments, the invention is not limited to only sugar free or diol free compositions, but can be a physical admixture of bortezomib and mannitol.

Aspects of the invention provide pharmaceutical compositions comprising bortezomib and one or more organic solvents. In embodiments, the organic solvent is an alcohol, including, without limitation, ethanol and t-butanol.

Suitable alcohols used as solvents for lyophilization include, for example, primary, secondary, and tertiary alcohols (e.g., ethanol, isopropyl alcohol, and t-butyl alcohol). In embodiments, an alcohol is a sterically hindered alcohol, such as t-butyl alcohol; however, any suitable organic solvents can be used in the invention.

Other solvents that can be used comprise dimethylacetamide, dimethylisosorbide, dimethylsulfoxide, N-methylpyrrolidone, and combinations thereof.

In embodiments, the composition of may include from about 1% to about 100% by volume of organic solvent.

Compositions of the present invention may further comprise water, in addition to an organic solvent. An aqueous organic solvent mixture typically comprises from about 5% to about 95% by volume of organic solvent.

Also provided are processes for the preparation of injectable pharmaceutical formulations which may be in the form of ready-to-use solutions or lyophilized preparation or a physical admixture.

An aspect of the present invention provides pharmaceutically stable preparations of bortezomib comprising bortezomib or a pharmaceutically acceptable salt thereof, and optionally a pharmaceutically acceptable carrier;

wherein the preparation is stable for at least one week when stored at 60° C. in a closed container, or at other temperature and relative humidity (“RH”) conditions.

Bortezomib when lyophilized alone, using t-butyl alcohol as a solvent for lyophilization, yields a stable lyophilized bortezomib, which can be analyzed for purity levels, including determinations of highest single impurity as well as total impurities.

Injectable formulations of the present invention can be prepared according to conventional freeze-drying or lyophilization techniques, including use of nitrogen flush as the blanket on the substance to be lyophilized or using a lyophilizer. The pH of the final preparation is adjusted to a desired value by adding an acid or base, as appropriate. Injectable formulations also can be subjected to terminal sterilization steps in the manufacturing process and can be lyophilized and filled into containers such as glass vials.

In certain embodiments, ready-to-use solutions of bortezomib comprise lyophilized bortezomib dissolved in water for injection or any other suitable solvent such as saline solutions and the like, which can be injected directly without any reconstitution step.

In all of the embodiments, the mixtures may further comprise one or more pharmaceutically acceptable excipients, carriers, diluents, fillers, salts, buffers, stabilizers, solubilizers, and other materials known in the art. The preparation of pharmaceutically acceptable formulations comprising these materials is described in, e.g., A. Gennaro, Ed., Remington's Pharmaceutical Sciences, 18th Edition, Mack Publishing Co., Easton, Pa., 1990.

Certain compositions for oral administration are administered in pharmaceutically suitable solid forms prepared using conventional methods, for immediate release of the pharmaceutically active compound. They may also be formulated so as to provide delayed or controlled release of the active ingredient therein using release retarding polymers in varying proportions to provide the desired release profile.

Therapeutically effective amounts of active ingredient can be provided in the form of pharmaceutical formulations in the form of tablets, capsules, granules (synonymously, “beads” or “particles” or “pellets”), suspensions, emulsions, powders, dry syrups, and the like. All such formulations are included herein without limitation.

During the processing of the oral dosage forms, one or more pharmaceutically acceptable excipients may optionally be included, such as but not limited to any one or more of diluents, binders, disintegrants, lubricants, glidants, coloring agents, film-forming agents, and others.

### Diluents:

Various useful fillers or diluents include, but are not limited to, starches, lactose, mannitol (Pearlitol™ SD200), cellulose derivatives, confectioner's sugar and the like. Different grades of lactose include but are not limited to lactose monohydrate, lactose DT (direct tableting), lactose anhydrous, Flowlac™ (available from Meggle Products), Pharmatose™ (available from DMV) and others. Different starches include, but are not limited to, maize starch, potato starch, rice starch, wheat starch, pregelatinized starches (commercially available as PCS PC10 from Signet Chemical Corporation), and starch 1500, starch 1500 LM grade (low moisture content grade) from Colorcon, fully pregelatinized starches (commercially available as National 78-1551 from Essex Grain Products) and others. Different cellulose compounds that can be used include crystalline celluloses and powdered celluloses. Examples of crystalline cellulose products include but are not limited to CEOLUS™ KG801, Avicel™ PH101, PH102, PH301, PH302 and PH-F20, PH-112 microcrystalline cellulose 114, and microcrystalline cellulose 112, silicified microcrystalline celluloses (e.g., Prosolv™ supplied by JRS Pharma). Other useful diluents include but are not limited to carmellose, sugar alcohols such as mannitol (Pearlitol™ SD200), sorbitol and xylitol, calcium carbonate, magnesium carbonate, dibasic calcium phosphate, and tribasic calcium phosphate.

### Binders:

Various useful binders include, but are not limited to, hydroxypropyl celluloses, also called HPC (Klucel™ LF, Klucel EXF) and useful in various grades, hydroxypropyl methylcelluloses, also called hypromelloses or HPMC (Methocel™) and useful in various grades, polyvinylpyrrolidones or povidones (such as grades PVP-K25, PVP-K29, PVP-K30, and PVP-K90), Plasdone™ S 630 (copovidone), powdered acacia, gelatin, guar gum, carbomers (Carbopol™ products), methylcelluloses, polymethacrylates, and starches.

### Disintegrants:

Various useful disintegrants include, but are not limited to, carmellose calcium (Gotoku Yakuhin Co., Ltd.), carboxymethylstarch sodium (Matsutani Kagaku Co., Ltd., Kimura Sangyo Co., Ltd., etc.), croscarmellose sodium (Ac-di-sol™ from FMC-Asahi Chemical Industry Co., Ltd.), crospovidones, examples of commercially available crospovidone products including but not limited to crosslinked povidone, Kollidon™ CL [manufactured by BASF (Germany)], Polyplasdone™ XL, XI-10, and INF-10 [manufactured by ISP Inc. (USA)], and low-substituted hydroxypropylcelluloses. Examples of low-substituted hydroxypropylcellulose include but are not limited to low-substituted hydroxypropylcellulose LH11, LH21, LH31, LH22, LH32, LH20, LH30, LH32 and LH33 (all manufactured by Shin-Etsu Chemical Co., Ltd.). Other useful disintegrants include sodium starch glycolate, colloidal silicon dioxide, and starches.

### Lubricants:

An effective amount of any pharmaceutically acceptable tableting lubricant can be added to assist with compressing tablets. Useful tablet lubricants include magnesium stearate, glyceryl monostearates, palmitic acid, talc, carnauba wax, calcium stearate sodium, sodium or magnesium lauryl sulfate, calcium soaps, zinc stearate, polyoxyethylene monostearates, calcium silicate, silicon dioxide, hydrogenated vegetable oils and fats, stearic acid and combinations thereof.

### Glidants:

One or more glidant materials, which improve the flow of powder blends and minimize dosage form weight variations can be used. Useful glidants include, but are not limited to, silicon dioxide, talc and combinations thereof.

### Coloring Agents:

Coloring agents can be used to color code the compositions, for example, to indicate the type and dosage of the therapeutic agent therein. Suitable coloring agents include, without limitation, natural and/or artificial compounds such as FD&C coloring agents, natural juice concentrates, pigments such as titanium oxide, iron oxides, silicon dioxide, and zinc oxide, combinations thereof, and the like.

### Film-Forming Agents:

Various film-forming agents that are useful for coating dosage forms include, but are not limited to, cellulose derivatives such as soluble alkyl- or hydroalkyl-cellulose derivatives such as methyl celluloses, hydroxymethyl celluloses, hydroxyethyl celluloses, hydroxypropyl celluloses, hydroxymethylethyl celluloses, hydroxypropyl methylcelluloses, sodium carboxymethyl celluloses, etc., insoluble cellulose derivative such as ethyl celluloses and the like, dextrins, starches and starch derivatives, polymers based on carbohydrates and derivatives thereof, natural gums such as gum Arabic, xanthans, alginates, polyacrylic acid, polyvinyl alcohols, polyvinyl acetates, polyvinylpyrrolidones, polymethacrylates and derivatives thereof (Eudragit™ products), chitosan and derivatives thereof, shellac and derivatives thereof, waxes and fat substances. Useful enteric coating materials include but are not limited to materials such as cellulosic polymers like cellulose acetate phthalates, cellulose acetate trimellitates, hydroxypropyl methylcellulose phthalates, polyvinyl acetate phthalates, etc., methacrylic acid polymers and copolymers (Eudragit™), and the like, and mixtures thereof.

Certain excipients are frequently used as adjuvants for the coating processes, including plasticizers, opacifiers, antiadhesives, polishing agents, etc. Various useful plasticizers include but are not limited to castor oil, diacetylated monoglycerides, dibutyl sebacate, diethyl phthalate, glycerin, polyethylene glycol, propylene glycol, triacetin, triethyl citrate, and mixtures thereof. An opacifier like titanium dioxide may also be present in an amount ranging from about 10% (w/w) to about 20% (w/w) based on the total weight of the coating.

Antiadhesives are frequently used in film coating processes to avoid sticking effects during film formation and drying. An example of a useful antiadhesive for this purpose is talc. The antiadhesive is frequently present in the film coating in an amount of about 5% (w/w) to 15% (w/w) based upon the total weight of the coating.

Suitable polishing agents include polyethylene glycols of various molecular weights or mixtures thereof, talc, surfactants (e.g. glycerol monostearate and poloxamers), fatty alcohols (e.g., stearyl alcohol, cetyl alcohol, lauryl alcohol and myristyl alcohol) and waxes (e.g., carnauba wax, candelilla wax and white wax).

When coloured tablets are desired, the colour is normally applied in the coating. Consequently, colouring agents and pigments may be present in the film coating. Various colouring agents include but not limited to iron oxides, which can be red, yellow, black or blends thereof.

In addition to the above coating ingredients, sometimes pre-formulated coating products such as OPADRY™ products (supplied by Colorcon) or TABCOAT™ products can be used. OPADRY compositions generally comprise polymer, plasticizer and, if desired, pigment in a dry concentrate. OPADRY products produce attractive, elegant coatings on a variety of tablet cores and can be used in both aqueous and organic coating procedures. Products sold in a dry form generally require only dispersion in a liquid before use.

Polymers that can be used in the present invention include hydrophilic and hydrophobic substances, and combinations thereof. Suitable polymers include, but are not limited to, cellulose ethers, e.g., hydroxypropyl methylcelluloses or hypromelloses (HPMC), ethylcelluloses, hydroxypropylcelluloses (HPC), hydroxyethylcelluloses and carboxymethylcellulose sodium, polyvinylpyrrolidones, including noncross-linked polyvinylpyrrolidones, carboxymethylstarch, polyethylene glycols, polyoxyethylenes, poloxamers (polyoxyethylene-polyoxypropylene copolymers), polyvinylalcohols, glucanes (glucans), carrageenans, scleroglucanes (scleroglucans), mannans, galactomannans, gellans, alginic acid and derivatives (e.g., sodium or calcium alginate, propylene glycol alginate), polyaminoacids (e.g., gelatin), methyl vinyl ether/maleic anhydride copolymers, polysaccharides (e.g., carageenan, guar gum, xanthan gum, tragacanth and ceratonia), alpha-, beta-, or gamma-cyclodextrins, and dextrin derivatives (e.g., dextrin), polymethacrylates (e.g., copolymers of acrylic and methacrylic acid esters containing quaternary ammonium groups), cellulose esters (e.g. cellulose acetate), acrylic acid polymers (e.g., carbomers), chitosan and derivatives thereof, and shellac and derivatives thereof.

Sugar coating can also be performed using any process and excipients as are known to the person skilled in the art.

Equipment suitable for processing the pharmaceutical compositions of the present invention include rapid mixer granulators, planetary mixers, mass mixers, ribbon mixers, fluid bed processors, mechanical sifters, homogenizers, blenders, roller compacters, extrusion-spheronizers, compression machines, capsule filling machines, rotating bowls or coating pans, tray dryers, fluid bed dryers, rotary cone vacuum dryers, and the like, multimills, fluid energy mills, ball mills, colloid mills, roller mills, hammer mills, and the like, equipped with a suitable screen.

The formulations are prepared using bortezomib particles having mean particle sizes of about 1 μm to about 200 μm, about 3 μm to about 100 μm, or about 5 μm to about 50 μm. Such particles of the active ingredient exhibit required micromeritic properties such as, but not limited to, bulk density, tapped density, angle of repose, Carr index, compressibility ratio, and the like.

As used herein, the term “mean particle size” refers to a distribution of particles wherein about 50 volume percent of all particles measured have particle sizes less than the defined mean particle size value, and about 50 volume percent of all measurable particles measured have particle sizes greater than the defined mean particle size value; this can be denoted by the term “D₅₀.” Similarly, a particle size distribution where 90 volume percent of the particles have sizes less than a specified size is referred to as “D₉₀” and a distribution where 10 volume percent of particles have sizes less than a specified size is referred to as “D₁₀.” A desired particle size range material can be obtained directly from a synthesis process or any known particle size reduction processes can be used, such as but not limited to sifting, milling, micronization, fluid energy milling, ball milling, and the like. Methods for determining D₁₀, D₅₀ and D₉₀ include laser light diffraction, such as using equipment from Malvern Instruments Ltd. (Malvern, Worcestershire, United Kingdom).

The invention includes the use of packaging materials such as containers and closures of high-density polyethylene (HDPE), low-density polyethylene (LDPE) and or polypropylene and/or glass, glassine foil, aluminum pouches, and blisters or strips composed of aluminum or high-density polypropylene, polyvinyl chloride, polyvinylidene dichloride, etc.

Vials are small, usually glass, containers that can be sealed with a suitable stopper and seal, and other suitable primary containers may be used, for instance, but not limited to, pre-filled syringes. Vials also are sealed containers of medication that are used once and include breakable and non-breakable closed glass containers, breakable plastic containers, miniature screw-top jars, and any other type of container of a size capable of holding only one unit dose of a drug (typically in a volume no more than about 5 mL).

A lyophilized bortezomib formulation is contained within a container that is sealed aseptically. The container is provided with an opening and a means for aseptically sealing the opening, e.g., such that the sealed container is fluidly sealed or the sealed opening is substantially impermeable to atmospheric gases, moisture, pathogenic microorganisms, or the like. The container can be constructed of any suitable material such as, for example, glass, polypropylene, Dalkyo Resin CZ (sold by Dalkyo Gomu Seiko, Ltd.), polyethylene terephthalate, and the like. In embodiments, the container is constructed of glass. Suitable glass containers include, but are not limited to, glass vials. Suitable glass vials include molded and tubing glass vials such as, for example, Type I molded glass vials, and the like. Such molded and tubing glass vials can be obtained from Kimble Glass, Inc., Vineland, N.J., Wheaton Science Products, Millville, N.J., or other companies. Preferably, the container contains a therapeutically effective dose of the lyophilized bortezomib formulation and is of sufficient volume (i.e., has sufficient capacity) to contain the volume of liquid that will be used for reconstitution.

A suitable means for sealing the container can include, for example, a stopper, a cap, a lid, a closure, a covering which fluidly seals the container, or the like. The means for sealing the container are not limited to separate closures or closure devices. The means for aseptically sealing the container includes a stopper such as, for example, a stopper that is configured to fluidly seal the opening. Suitable stoppers include conventional medical grade stoppers that do not degrade or release significant amounts of impurities upon exposure to the reconstituted aqueous bortezomib solution. In embodiments, the stopper is constructed of an elastomer, such as an elastomer that is pierceable by a hypodermic needle or a blunt cannula. Exemplary stoppers include 6720 GC gray rubber stoppers from American Stelmi Corporation, 4432/50 gray rubber stoppers from West Company, and the like.

Optionally, an outer seal is provided to cover and entirely surround the stopper. The outer seal can be constructed of any suitable material. When an outer seal is used, it can be fitted with a lid that can be easily manually removed to provide access to the stopper. Suitable outer seals can include, for example, flip-off aluminum/polypropylene seals (lacquered or non-lacquered aluminum), such as are marketed by The West Company, Inc., and other manufacturers. Such seals include an outer rim made of a suitable material, such as aluminum, that entirely surrounds the lateral edge of the stopper and further include a lid (typically polypropylene or other suitable material) that entirely covers the upper surface of the stopper. The polypropylene lid can be “flipped” off, e.g., by exerting upward pressure with a finger or thumb, to provide access to the stopper, so that it can be punctured with a hypodermic needle to deliver an aqueous vehicle for constitution. Optionally, the seal can be removed in its entirety to allow the powder to be poured from the vial.

The compositions according to aspects of the invention mentioned above can be readily reconstituted by adding an aqueous solvent. The reconstitution solvent is suitable for pharmaceutical administration. Examples of suitable reconstitution solvents include, without limitation, water, saline, and phosphate buffered saline (PBS). For clinical use, the compositions are frequently reconstituted with sterile saline (0.9% NaCl w/v).

Mention of bortezomib is intended to include any of the alternative forms in which bortezomib can be administered, such as salts, esters, hydrates, solvates, crystalline or amorphous polymorphs, racemic mixtures, enantiomeric isomers, etc.

The following examples further describe certain specific aspects and embodiments of the invention and demonstrate the practice and advantages thereof. It is to be understood that the examples are given for purposes of illustration only and are not intended to limit the scope of the invention in any manner.

### Example 1

### Bortezomib Formulation

| Ingredient | mg/Vial |
|---|---|
| Bortezomib | 3.5 |
| Amino acid, vitamin, carboxylic | 35 |
| acid, or sodium chloride | |

A physical admixture is prepared by mixing powdered bortezomib and amino acid or vitamin or carboxylic acid or sodium chloride manually and then the mixture is filled into a vial. The physical admixture is dissolved in sterile water for injection prior to use.

A lyophilized preparation can also be prepared using the following manufacturing process:

1. Dissolve the amino acid, vitamin, carboxylic acid, or sodium chloride in water for Injection.

2. Dissolve bortezomib in the solution.

3. Filter the solution through a 0.2 μm sterile membrane filter.

4. Fill the filtrate into the depyrogenated USP type I glass vials and loosely stopper the vials.

5. Lyophilize the loosely stoppered vials in a freeze dryer.

6. After lyophilization, stopper the vials completely by hydraulic pressing and seal the vials with flip-off seals.

The lyophilized product is reconstituted using sterile water for injection prior to use.

### Example 2

### Bortezomib Formulation

| Ingredient | mg/Vial |
|---|---|
| Bortezomib | 3.5 |
| Amino acid, vitamin, carboxylic | 35 |
| acid, or sodium chloride | |
| EDTA | 4 |

Manufacturing Procedure:

A physical admixture is prepared by combining powdered bortezomib and amino acid, vitamin, carboxylic acid, or sodium chloride, and optionally EDTA, manually and the mixture is filled into a vial. The physical admixture is dissolved in sterile water for injection prior to use.

A lyophilized preparation can also be prepared using the following manufacturing process:

1. Dissolve the amino acid, vitamin, carboxylic acid, or sodium chloride, and optionally EDTA, in water for injection.

2. Dissolve bortezomib in the solution.

3. Filter the solution through a 0.2 μm sterile membrane filter.

4. Fill the filtrate into depyrogenated USP type I glass vials and loosely stopper the vials.

5. Lyophilize the loosely stoppered vials in a freeze dryer.

6. After lyophilization, stopper the vials completely by hydraulic pressing and seal the vials with flip-off seals.

The lyophilized product is reconstituted using sterile water for injection prior to use.

### Example 3

### Ready-to-Use Solution Bortezomib Formulation

| Ingredient | Quantity/Vial | |
|---|---|---|
| Bortezomib | 3.5 | mg |
| Amino acid, vitamin, carboxylic | 35 | mg |
| acid, sodium chloride, or EDTA | | |
| Water for injection | 3.5 | mL |

Manufacturing Process:

Dissolve bortezomib and amino acid, vitamin, carboxylic acid, sodium chloride, or EDTA in water and fill the solution into a vial.

### Example 4

### Ready-to-Use Solution Bortezomib Formulation

| Ingredient | Quantity/Vial | |
|---|---|---|
| Bortezomib | 3.5 | mg |
| Water for injection | 3.5 | mL |

Manufacturing Process:

Bortezomib is dissolved in water and the solution is filled into a vial.

### Example 5

### Parenteral Bortezomib Composition with Cyclodextrin

| Ingredient | mg/Vial |
|---|---|
| Bortezomib | 3.5 |
| Cyclodextrin | 14 |

Manufacturing Procedure:

A physical admixture is prepared by combining powdered bortezomib and cyclodextrin manually and then the mixture is filled into a vial. The physical admixture is dissolved in sterile water for injection prior to use.

A ready-to-use solution is prepared by dissolving bortezomib and cyclodextrin in water for injection and the solution is filled into a vial. The solution can be directly injected without any further dilution prior to use.

A lyophilized formulation is prepared using the following manufacturing process:

1. Dissolve bortezomib and cyclodextrin in water for injection.

2. Filter the solution through a 0.2 μm sterile membrane filter.

3. Fill the filtrate into depyrogenated USP type 1 glass vials and loosely stopper the vials.

4. Lyophilize the loosely stoppered vials in a freeze dryer.

5. After lyophilization, stopper the vials completely by hydraulic pressing and seal the vials with flip-off seals.

The lyophilized product is reconstituted using sterile water for injection prior to use.

### Example 6

### Oral Bortezomib Composition with Cyclodextrin

| Ingredient | mg/Dose |
|---|---|
| Bortezomib | 3.5 |
| Cyclodextrin | 28 |

Manufacturing Process:

Bortezomib and cyclodextrin are added to an appropriate volume of an organic solvent or water. This solution is filtered through a 0.2 μm sterile membrane and then evaporated under sterile conditions to dryness at room temperature under reduced pressure, to yield a white to off-white solid. The solid is crushed under sterile conditions, to yield a free-flowing powder. The free-flowing powder is mixed with pharmaceutically acceptable excipients and compressed into tablets for oral administration.

### Example 7

### Parenteral Bortezomib Composition with a Solubilizer

| Ingredient | mg/Vial |
|---|---|
| Bortezomib | 3.5 |
| Propylene glycol monocaprylate | 250 |

A physical admixture is prepared by combining powdered bortezomib and propylene glycol monocaprylate manually, and the mixture is filled into a vial. The physical admixture is dissolved in sterile water for injection prior to use.

A ready-to-use solution is prepared by dissolving bortezomib and Propylene glycol monocaprylate in water for injection and the solution is filled into a vial. The solution can be directly injected without any further dilution prior to use.

A lyophilized formulation is prepared using the following manufacturing process:
1. Dissolve Propylene glycol monocaprylate in water for injection.
2. Dissolve bortezomib in a water-miscible organic solvent.
3. Mix the solutions and stir until a clear solution is obtained.
4. Filter the solution through a 0.2 μm sterile membrane filter.
5. Fill the filtrate into depyrogenated USP type 1 glass vials and loosely stopper the vials.
6. Lyophilize the loosely stoppered vials in a freeze dryer.
7. After lyophilization, stopper the vials completely by hydraulic pressing and seal the vials with flip-off seals.

The lyophilized product is reconstituted using sterile water for injection prior to use.

### Example 8

### Oral Bortezomib Composition with a Solubilizer

| Ingredient | mg/Dose |
|---|---|
| Bortezomib | 3.5 |
| Propylene glycol monocaprylate | 500 |

Manufacturing Process:

Bortezomib and propylene glycol monocaprylate are combined with an appropriate volume of an organic solvent or water. This solution is filtered through a 0.2 μm sterile membrane and then evaporated under sterile conditions to dryness at room temperature under reduced pressure, to yield a white to off-white solid. This solid is then crushed under sterile conditions to yield a free-flowing powder. The free-flowing powder is mixed with pharmaceutically acceptable excipients and compressed into tablets for oral administration.

### Example 9

### Lyophilized Bortezomib Formulation

| Ingredient | mg/Vial |
|---|---|
| Bortezomib | 3.5 |
| t-Butyl alcohol | q.s. |

Manufacturing Process:

1. Dissolve bortezomib in t-butyl alcohol.

2. Filter the solution through a 0.2 μm sterile membrane filter.

3. Fill the filtrate into a depyrogenated glass vial and loosely stopper the vial.

4. Lyophilize the loosely stoppered vial in a freeze dryer.

5. Fill the head space of the vial after freeze drying with an inert gas and then stopper the vial completely by hydraulic pressing and seal with a flip-off seal

The lyophilized product is reconstituted using a suitable solvent prior to use.

The impurity content of packaged samples of a lyophilized bortezomib composition, exposed to different temperature and relative humidity (RH) storage conditions, is compared with a commercial composition (VELCADE®), as received. A lyophilized bortezomib preparation is comparable to the commercial product in impurity levels, as shown in Table 1. The impurity percentages are percentages of the label bortezomib content.

TABLE 1

| | | Parameter | | |
|---|---|---|---|---|
| | | | Highest | Total |
| | Exposure | Physical | Impurity | Impurities |
| Sample | Conditions | Description | (%) | (%) |
| Lyophilized | 25 ± 2° C. and | White to off- | 0.05 | 0.1 |
| formulation (3.5 mg | 60 ± 5% RH for 1 | white powder | | |
| of bortezomib | Week | | | |
| dissolved in 5 mL | 40 ± 2° C. and | White to off- | 0.06 | 0.1 |
| of t-butyl alcohol, | 75 ± 5% RH for 1 | white powder | | |
| and lyophilized to | Week | | | |
| remove solvent) | 60° C. for 1 Week | White to off- | 0.05 | 0.4 |
| | | white powder | | |
| VELCADE ® Batch | As received | White to off- | 0.07 | 0.29 |
| No. BC001A1, | | white powder | | |
| Expiration: January | | | | |
| 2010 | | | | |

The comparative study of lyophilized bortezomib preparation and a commercial product shows that the highest single impurity and total impurities are below 0.5%. The stress stability data indicate the feasibility of label storage conditions for the product as “store below 25° C.”

### Example 10

### Physical Bortezomib Admixture

| Ingredient | mg/Vial |
|---|---|
| Bortezomib | 3.5 |
| Amino acid, vitamin, carboxylic | 35 |
| acid, or sodium chloride | |

Manufacturing Process:

A physical admixture is prepared by combining powdered lyophilized bortezomib (prepared using a process as described in Example 9) and optionally an amino acid, vitamin, carboxylic acid, or sodium chloride, and then the mixture is filled into a vial. The physical admixture is dissolved in a suitable solvent prior to use.

### Example 11

### Ready-to-Use Bortezomib Solution Formulation

| Ingredient | Quantity/Vial | |
|---|---|---|
| Bortezomib | 3.5 | mg |
| Water for injection | 3.5 | mL |

Manufacturing Process:

Lyophilized bortezomib, prepared as described in Example 9, is dissolved in water or any other suitable solvent and the solution is filled into a vial. The solution can be directly used without any reconstitution step.

### Example 12

### Ready-to-Use Bortezomib Formulation

| Ingredient | mg/Vial |
|---|---|
| Bortezomib | 3.5 |
| Dimethylsulfoxide | 1000 |

Manufacturing Process:

1. Dissolve bortezomib in dimethylsulfoxide.

2. Sterile filter and aseptically fill the desired volume of solution into a glass or plastic syringe.

A desired volume of solution can also be filled into a glass or plastic vial.

1. A pharmaceutical formulation in a liquid state, comprising bortezomib or a pharmaceutically acceptable salt thereof and at least one organic solvent.

2. The pharmaceutical formulation of claim 1, wherein an organic solvent comprises an alcohol, N,N-dimethylacetamide, dimethylisosorbide, dimethylsulfoxide, N-methylpyrrolidone, or a combination of any two or more thereof.

3. The pharmaceutical formulation of claim 1, further comprising water.

4. The pharmaceutical formulation of claim 1, wherein an organic solvent is a primary, secondary, or tertiary alcohol.

5. The pharmaceutical formulation of claim 1, comprising from about 1 to about 100 percent by volume of organic solvent.

6. The pharmaceutical formulation of claim 1, further comprising one or more of pharmaceutically acceptable solubilizers and stabilizers.

7. The pharmaceutical formulation of claim 1, the form of a ready-to-use solution.

8. The pharmaceutical formulation of claim 1, packaged in a glass vial, plastic vial, or pre-filled syringe.

9. A solid pharmaceutical formulation, comprising a liquid of claim 1 after being lyophilized to form a solid.

10. A sugar free pharmaceutical formulation, comprising bortezomib, or a pharmaceutically acceptable salt thereof, and a pharmaceutically acceptable carrier.

11. The pharmaceutical formulation of claim 10, wherein a pharmaceutically acceptable carrier comprises at least one of an amino acid, a vitamin, a carboxylic acid, and sodium chloride.

12. The pharmaceutical formulation of claim 10, wherein a pharmaceutically acceptable carrier further comprises a stabilizing agent.

13. The pharmaceutical formulation of claim 10, further comprising ethylenediaminetetraacetic acid or a salt thereof.

14. The pharmaceutical formulation of claim 10, wherein bortezomib and a pharmaceutical carrier are present in molar ratios ranging from about 0.5:1 to about 100:1.

15. The pharmaceutical formulation of claim 10, in the form of a ready-to-use solution, a lyophilized solid, or a solid physical admixture.

16. The pharmaceutical formulation of claim 10, further comprising a pH adjusting agent.

17. A pharmaceutical formulation, comprising bortezomib, or a pharmaceutically acceptable salt thereof, and a pharmaceutically acceptable carrier comprising at least one of cyclodextrin and a solubilizer.

18. The pharmaceutical formulation of claim 17, wherein a cyclodextrin comprises an alpha-, beta-, or gamma-cyclodextrin, or a derivative thereof.

19. The pharmaceutical formulation of claim 17, wherein a solubilizer comprises at least one of a surface active agent, a co-solvent, and a complexing agent.

20. The pharmaceutical formulation of claim 17, wherein bortezomib and a pharmaceutically acceptable carrier are present in a molar ratio ranging from about 0.5:1 to about 100:1.

21. The pharmaceutical formulation of claim 17, in a solid form for oral administration.

22. The pharmaceutical formulation of claim 17, in lyophilized form or a physical admixture, for reconstitution with a liquid.

23. A solid pharmaceutical formulation, prepared by lyophilizing a solution comprising bortezomib and an alcohol.

24. The solid pharmaceutical formulation of claim 23, wherein an alcohol comprises t-butyl alcohol.

25. The solid pharmaceutical formulation of claim 23, wherein a solution consists essentially of bortezomib and t-butyl alcohol.
